(19) United States
(12) Patent Application Publication
(10) Pub. No.: US 2007/0148260 A1
(43) Pub. Date: Jun. 28, 2007

(54) Method for maintaining cerebral hemispheric oxygen saturation during surgery

(76) Inventor: Andre Denault, Longueuil (CA)

Correspondence Address: Louis Tessier, P.O. Box 54029, Town of Mount-Royal, QC, H3P 3H4, US

(21) Appl. No.: 11/453,026

(22) Filed: Jun. 15, 2006

Related U.S. Application Data
(60) Provisional application No. 60/752,366, filed on Dec. 22, 2005.

(51) Int. Cl. A61K 31/21; A61K 33/00

(52) U.S. Cl. 424/608; 514/509

(57) ABSTRACT

A method for preventing or reducing hemispheric cerebral oxygen desaturation in a subject undergoing surgery, wherein the method comprising the prophylactic administration of a vasodilator to the subject.

This application claims priority from U.S. Provisional Patent Applications Ser. No. 60/752,366 filed Dec. 22, 2005, the disclosure of which is hereby incorporated by reference in its entirety.

### FIELD OF THE INVENTION

The present invention relates to a method for maintaining cerebral hemispheric oxygen saturation during surgery. Specifically, the present invention comprises the use of a vasodilator administered to a subject to prevent or reduce cerebral hemispheric oxygen desaturation.

### BACKGROUND OF THE INVENTION

Cardiopulmonary bypass (CPB) has long been recognized as a major contributor to the inflammatory response observed after cardiac surgery (1-3). One of the proposed mechanisms of this reaction is endothelial activation and dysfunction, induced by ongoing ischemic-reperfusion episodes during the intense physiologic stress of CPB. These mechanisms are associated with many clinical complications observed after cardiac surgery, one of them being adverse neurological outcome (4-6).

Traditionally, NTG has been used in cardiac surgery to control blood pressure control (24-26). As a nitric oxide (NO) donor, independent from endogenous NO synthetase (27), it causes a local vasodilatation, but may also inhibit platelet aggregation (28) and neutrophilic adhesion. Apart from its well-known hemodynamic effects, NTG has been studied as a preventive measure to decrease perioperative myocardial ischemia (25, 29-33). More recently, the protective effect of NTG in ischemic-reperfusion animal models (8-12) has been explored with promising results. One of the rare human clinical trials on this topic (13) studied the risk of developing acute respiratory distress syndrome (ARDS) in a group of patients submitted to very high-risk surgeries (estimated risk of ARDS of 10%). In this clinical study, the investigators did not observe any post-operative cases of ARDS among the 56 patients treated with high-dose intravenous NTG (1 to 5 μg/kg/min), as compared to 17% in the control group of 24 patients. The intravenous NTG group had better transcutaneous oxygen pressure as a marker of tissue perfusion. However the investigators were not blinded to the use of NTG.

A retrospective study by Goldman et al. (7) involved the use of intravenous NTG to maintain cerebral oxygen saturation near preoperative baseline values during on-pump and off-pump surgeries, and showed that it was possible to lower the incidence of permanent strokes in the study group (<1%), compared to an historical control group (2%), even when adjusted for the type of surgery (on-pump vs off-pump). As a nitric oxide donor, nitroglycerine (NTG) could provide a mechanism of protection against ischemic-reperfusion injuries, as shown in some animal studies (8-12), and could contribute to maintaining regional perfusion. However, these prior studies have only used NTG after oxygen saturation levels have already started to drop.

One reason that NTG may not have been administered to patients before oxygen saturation levels start to drop is that NTG is known a vasodilator and as such reduces arterial pressure. Therefore, those skilled in the art might expect it to be inappropriate or even harmful to administer NTG to a patient with normal cerebral oxygen saturation prior to a reduction in saturation levels, and particularly inappropriate prior to performing surgery.

In view of the above, there is a need in the industry to provide novel methods for maintaining cerebral hemispheric oxygen saturation during surgery.

### SUMMARY OF THE INVENTION

The present invention relates to a method for reducing or preventing hemispheric cerebral oxygen desaturation in a subject undergoing surgery. The method includes the prophylactic administration of a vasodilator to the subject. In one embodiment of the invention, the vasodilator is administered in an amount sufficient for maintaining hemispheric cerebral oxygenation above a predetermined oxygen saturation level. For example, and non-limitingly, the predetermined oxygen saturation level is equal to about 75 percent of an hemispheric cerebral oxygenation in the subject prior to the administration of the vasodilator and prior to the beginning of the surgery.

In the context of the present invention, “desaturation” refers to a reduced level of oxigenisation as compared to the baseline level measured before the start of surgery.

Suitable vasodilators that may be used in accordance with the present invention include those compounds that cause dilation or relaxation of the blood vessels, such as nitric oxide donors (e.g., nitroglycerine), nitroprusside or other vasodilators. In some embodiments fo the invention, the vasodilator is nitroglycerine.

The vasodilator may be administered to a subject intravenously (such as by injection or infusion), orally, or through the airways of the subject.

In yet another embodiment, the vasodilator may be administered intravenously (e.g., by injection) at a rate of about 0.01 μg/kg of subject weight/min to about 100 μg/kg of subject weight/min; of about 0.1 μg/kg of subject weight/min to about 5 μg/kg of subject weight/min; or of about 0.5 μg/kg of subject weight/min to about 1 μg/kg of subject weight/min.

In another embodiment of the invention, the vasodilator is administered only prior to performing the surgery. In other embodiments of the invention, the vasodilator is administered both prior to performing the surgery and during the surgery (i.e., during a portion of the surgery, or throughout the entire duration of the surgical procedure).

In another embodiment of the invention, the vasodilator is administered intravenously into the bloodstream of the subject at a first rate before the beginning of an extra-corporal circulation; and then administered intravenously into the bloodstream of the subject at a second rate after the beginning of the extra-corporal circulation. In yet another embodiment, the second rate is substantially larger than the first rate. For example, and non-limitingly, the second rate is about twice the first rate. However, in alternative embodiments of the invention, the vasodilator is administered in any other suitable manner.

The subject of the above-described methods may be a mammal, which includes both humans and non-humans (e.g., dogs, horses, cats, cows, pigs, among others).

Advantageously, the present method allows cerebral oxygen saturation to remain at suitable levels so as to reduce risks of mortality and morbidity in the subject during and after surgery. Also, many vasodilators suitable to perform the inventive method are readily available at relatively low cost.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

FIG. 1, in an X-Y graph view, illustrates mean right and left cerebral oxygen saturations from the time of anesthesia induction to the time of chest closure for patients undergoing a surgery involving extra-corporal circulation. The patients included a placebo group, and a vasodilated group that was administered nitroglycerine.

FIG. 2, in a schematic view, illustrates the treatment of hypotension before and during CPB according to a standardized protocol, using intravenous perfusion of neosinephrine or norepinephrine, and then, intravenous bolus of vasopressin, epinephrine or methylene blue.

FIG. 3, in a schematic view, illustrates a protocol used for weaning patients from CPB.

### DESCRIPTION OF THE PREFERRED EMBODIMENTS

Methods

The experiments described herein may be predictive of biological effects in humans or other mammals and/or may serve as models for use of the present invention in humans or other mammals for the prevention of a reduction in cerebral oxygen saturation during surgery (e.g., heart surgery, carotid artery surgery and any chest surgery, among other possibilities)

Study Population

Following approval by the ethics and research committees and written informed consent, 30 patients undergoing elective cardiac surgery in a tertiary care university hospital between March and November 2004 were recruited. To be eligible for the study, patients needed to undergo a cardiac surgical procedure requiring CPB and to be at high-risk as defined by a Parsonnet score>15 (19). Patients were excluded if they had received intravenous nitroglycerine (NTG) for more than 12 hrs within 24 hrs of surgery.

Treatment Protocol

All patients were premedicated with 0.1 mg/kg of morphine and 3-8 mg of midazolam administered intramuscularly approximately 1 hour before surgery. In the operating room, usual monitoring was installed, including 5-lead electrocardiogram, digital pulse saturometer, capnography, radial arterial line, a 15-cm 3-lumen catheter (CS-12703, Arrow International Inc., Reading, Calif.) and a pulmonary artery catheter (Swan-Ganz Thermodilution catheter 7.5 Fr; Baxter Healthcare Corporation, Irvine, Calif.). Regional cerebral oxygen saturation was monitored using near-infrared spectroscopy (INVOS 4100, Somanetics, Troy, Mich.) (20).

Near-infrared spectroscopy (NIRS) has been advocated as a useful monitor of brain oxygenation. It offers the advantage of providing assessment of regional cerebral oxygen saturation, even with a non-pulsatile flow, as during CPB. The NIRS oximeter has been validated against many other forms of cerebral monitoring (jugular venous saturation (14), cerebral blood flow (15)) and in many clinical contexts (cardiac surgery, neurosurgery, intensive care unit, etc (16-18)).

In the present study, a 5.0-MHz transesophageal echocardiographic omniplane probe (Hewlett-Packard Sonos 5500, Andover, Mass.) was then inserted and used as needed for cardiac and valvular function evaluation. Anesthesia was induced with an intravenous dose of 0.04 mg/kg of midazolam and 1 mg/kg of sufentanyl, and neuromuscular blockage was achieved with 0.6 mg/kg of rocuronium. Anesthesia was maintained with 1 mg/kg/h of sufentanyl, 0.04 mg/kg/h of midazolam, and 30-50 mg/kg/min of propofol. Isoflurane was used at need by the attending anesthesiologist.

All patients were ventilated with 100% oxygen and minute ventilation was adjusted to maintain a PaCO2 between 35-45 mmHg confirmed by serial arterial blood gas. Intravenous fluids (0.9% normal saline) were administered according to estimated insensible losses of 7 cc/kg/h. All patients received an intravenous bolus of aprotinine (2 MU), followed by a perfusion (500 000 U/hr) during CPB.

Nitroglycerin Administration Protocol

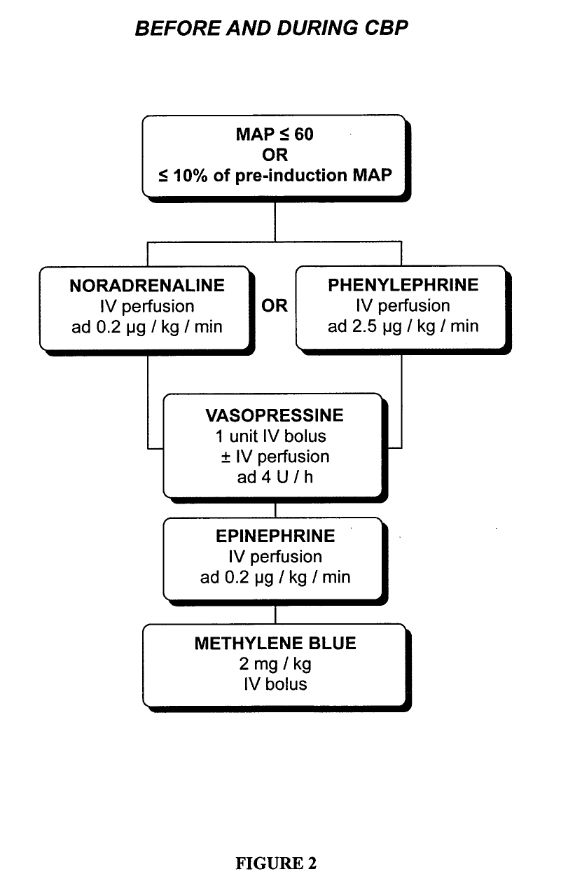

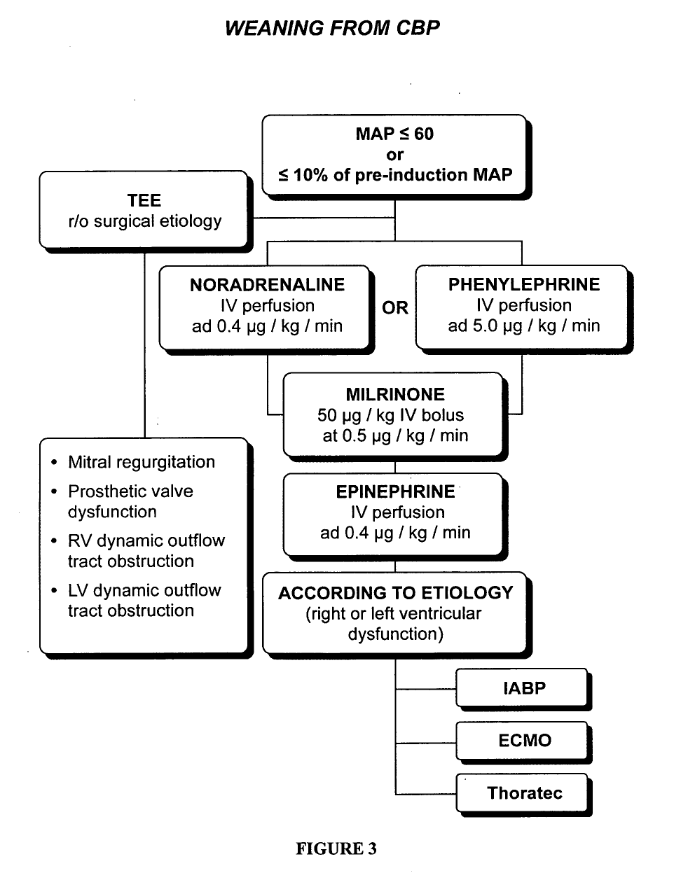

Randomization was done according to computerized random numbers. The study drug was prepared by the pharmacist and delivered to the operating room wrapped up in an opaque paper so that it was impossible for the anesthesiologist to know which perfusion was given to the patient. Following the induction of anesthesia, the administration of NTG (0.1 mg/mL; Sabex, Boucherville, QC, Canada) or placebo (D5%), began at a rate of 0.5 μg/kg/min, and was increased to 1 μg/kg/min immediately after the beginning of CPB. Based on previous studies, we used a NTG dose between 0.5 and 1 μg/kg/min. which would be adequate to prevent cardiac ischemia (21), safe (22, 23) and previously used to increase transcutaneous saturometry in high-risk surgery (13). At the end of the CPB, the study drug was stopped and the anesthesiologist was then free to use any useful medication (including intravenous NTG) for hemodynamic stabilization of the patient. Hypotension before or during CPB was treated according to a standardized protocol, using intravenous perfusion of neosinephrine or norepinephrine, and then, intravenous bolus of vasopressin, epinephrine or methylene blue (FIG. 2). In presence of refractory hypotension persisting more than 5 mins, the study drug was stopped. A protocol was used for weaning the patient from CPB (FIG. 3).

Data Collection

At the time of randomization, demographic, diagnostic (NYHA class, Parsonnet score, comorbidities, ejection fraction), and therapeutic (medication, type of surgery, redo) information was obtained for every patient.

After the induction of anesthesia and before the beginning of the study drug perfusion (time 0), baseline hemodynamic values (systemic and pulmonary arterial pressures, pulmonary arterial wedge pressure, heart rate, right atrial pressure, and cardiac output by standard thermodilution method) were measured along with arterial and mixed venous blood gas. The same values were recorded just before (time 1) and immediately after (time 2) CPB.

The cerebral oxygen saturation was recorded every 30 secs from the induction of anesthesia to the closure of the thorax. The CPB duration, aortic cross-clamping time, total intravenous fluids administered, total diuresis, total dose of heparin and total dose and duration of each vasopressor used were also recorded.

Outcome Measures

An outcome measure was observed based on mean and serial measures of hemispheric cerebral oxygen saturation during CPB. Other outcomes included other markers of tissue perfusion including whole blood lactate concentration, arteriovenous difference of partial CO2 pressure, and mixed venous oxygen saturation from time 0 to time 2; difficult separation from CPB, as defined as systolic arterial blood pressure lower than 80 mmHg with a diastolic pulmonary artery pressure or a wedge higher than 15 mmHg and use of vasopressors (norepinephrine>0.06 μg.kg-1.min-1, epinephrine>0.06 μg.kg-1.min-1, dobutamine>2 μg.kg-1.min-1), or use of intravenous milrinone during withdrawal of CPB or transport to the intensive care unit; other cardiac outcomes (CK-MB, use of a new intraaortic balloon pump during surgery, successful cardiopulmonary resuscitation during the hospital stay); and other clinical outcomes (length of ICU and total hospital stay, and death). Safety outcomes were also measured: blood losses during and 24 hrs after surgery, drop of hemoglobin during surgery, need for transfusion, and ratio of the partial pressure of oxygen in arterial blood to inspired O2 fraction (PaO2/FiO2 ratio), to explore any antiplatelet or ventilation effect. Follow-up ended when the patient was discharged from the hospital.

Statistical Analysis

The results are expressed as mean ± standard deviation or with median (min, max) according to the distribution for continuous variables, or as number and percentages for categorical variables. A logarithmic transformation was used when a continuous variable was not normally distributed.

For continuous variables, comparison of groups was performed using the parametric (t-test) or nonparametric (Wilcoxon) test depending on the distribution. For categorical variables, comparison of groups was performed using Pearson chi-square test.

Baseline hemodynamic values (systemic and pulmonary arterial pressures, pulmonary arterial wedge pressure, heart rate, right atrial pressure, and cardiac output by standard thermodilution method) were measured with arterial and mixed venous blood gas at times T0 (after the induction of anesthesia and before the beginning of the study drug perfusion), T1 (just before CPB) and T2 (immediately after CPB). To test variation between groups and over time, repeated measures ANOVA with GROUP, TIME (T0, T1 and T2) and GROUP×TIME interaction were performed. In case of statistically significant findings, appropriate contrasts were conducted, based on the global ANOVA model. Same method was used for analysis of repeated hemispheric cerebral saturation measures.

Statistical analysis was done with the computer software SAS version 8.02. A p value <0.05 was considered statistically significant.

Results

A total of 30 patients were enrolled in the study. Their clinical and demographic characteristics are presented in Table 1. Patients had a mean age of 73±10 years, a Parsonnet score of 27±9, and 67% had clinical congestive heart failure. Their mean baseline ejection fraction was 50±12%. Coronary artery bypass graft were performed in 5 patients, valvular procedures in 10, combined revascularization and valvular procedures surgeries in 15 and one patient had a Bentall procedure with a ventricular septal defect closure. Except for the use of beta-blockers on admission, which was higher in the NTG group (87% vs 47%; p=0.05), other initial characteristics were not statistically different among groups. Mean CPB time was 107±42 mins (97±32 mins in the control group vs. 118±49 mins in the treatment group; p=0.17). From the end of CPB to chest closure, patients in the control and the NTG groups received respectively 0.65±1.68 mg and 0.55±1.03 mg (p=0.55) of intravenous NTG.

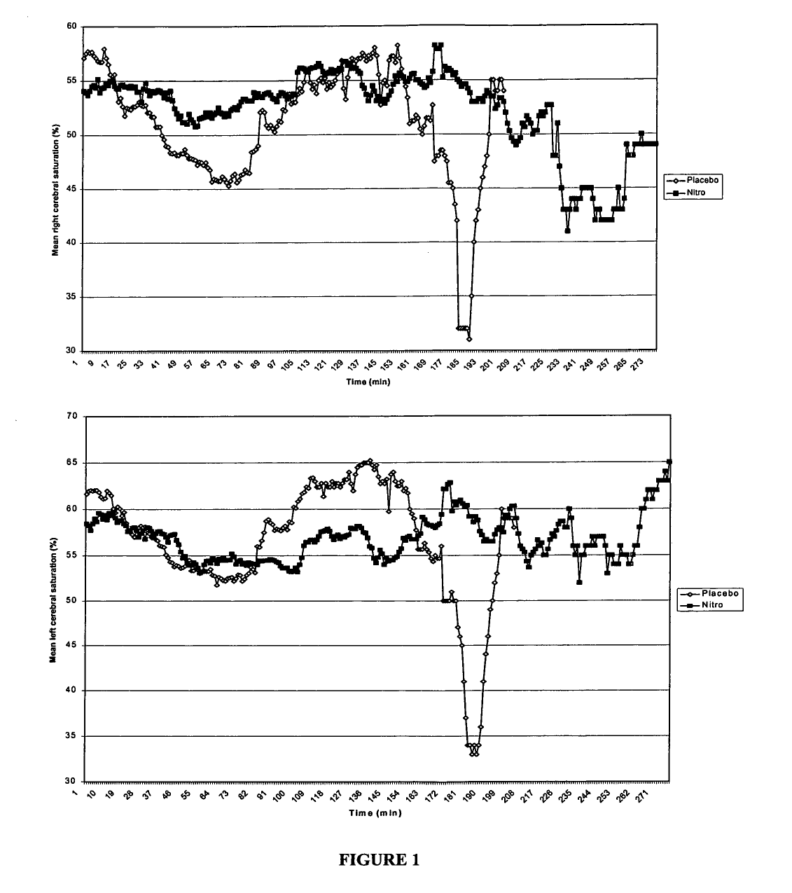

The evolution overtime of hemispheric cerebral oxygen saturation during the procedure was different in the NTG compared to the placebo group (Table 2 and FIG. 1). In the NTG group, both the left and right mean cerebral saturations were unchanged from the beginning to the end of the procedure as compared to the placebo group, in which the saturation decreased at the end of CPB (p=0.006, left; p=0.005, right) (FIG. 3). Respectively, 5 and 5 patients in the placebo group did not maintain their mean left and right saturations within 25% of their baseline, against 1 and 2 patients in the NTG group. Other indirect perfusion values (mixed venous blood oxygen saturation, arteriovenous difference of partial CO2 pressure and plasma lactates) did not show any statistically significant difference between groups (Table 2).

Both groups had similar hemodynamic profile (Table 3) although the right atrial pressures were slightly higher in the NTG group throughout the study (even before infusion) (p=0.03), as was the systolic pulmonary artery pressure (p=0.004). In both groups, the systolic blood pressure had a tendency to decrease from the induction of anesthesia to the beginning of the CPB, and to increase at the end of CPB (p=0.053). However there was no difference between groups. The heart rate, the right atrial pressure, the systolic pulmonary artery pressure, and the cardiac output were all significantly higher at the end of the CPB in both groups (p<0.05), compared to others values earlier in the surgery. As shown in Table 4, patients in the NTG group received more norepinephrine during the procedure (546±563 μg vs 1209±1037 μg; p=0.04). However, this difference was not statistically significant when expressed as a function of surgery length (p=0.096). There was no significant difference between groups in the amount of fluid infused (p=0.19). No patient in either group had their study drug stopped because of hypotension.

Other clinical outcomes are presented in Table 5. Patients in the NTG group had higher CK-MB the day after surgery (control: 19±12 vs NTG: 58±67, p=0.006). The proportion of hemodynamic instability at the end of CPB, the need for postoperative intra-aortic balloon pump (IABP), and the need for vasopressors for more than 24 hrs were the same in both groups. The hospital (but not the ICU) stay tended to be longer in the NTG group (14±7 vs 9±3, p=0.06). Two deaths occurred in NTG group. The first patient had a postoperative course complicated by a transient renal insufficiency and a cerebrovascular event (diagnosed on day 2), which kept her at the hospital until postoperative day 14. She was waiting for her transfer in a rehabilitation center, when she underwent sudden cardiac arrest with unsuccessful resuscitation. Because of an earlier episode of desaturation, the attending surgeon concluded to a probable pulmonary embolism. The second patient died on postoperative day 4. Prior to surgery, he was in NYHA class 4, had recent myocardial infarction, pulmonary hypertension, and a Parsonnet score of 30.5. His baseline left ventricular ejection fraction was 30%. At the end of a CPB of 83 mins, he required milrinone and norepinephrine perfusions, and an IABP. On postop day 1, a diagnosis of perioperative myocardial infarction was confirmed with 10-fold increase of CK-MB. Nevertheless, he was extubated and progressively weaned from vasopressors. The IABP was withdrawn on postoperative day 4 and the same evening, he suffered from a cardiopulmonary arrest. An autopsy revealed a global cardiac failure secondary to recent cardiac infarction, without other visible complications.

Table 5 also presents safety outcomes. Blood losses were similar in both groups during surgery (control: 429±261 vs NTG: 547±251, p=0.23), as were the transfused blood volumes (control: 332±408 vs NTG: 380±400, p=0.75) and the change of hemoglobin before and after surgery (p=0.17). Patients in the NTG group needed more heparin during surgery (control: 306±118 mg vs NTG: 393±111, p=0.047) but the same amount when corrected for CPB duration. The NTG group lost more blood during the first 24 hrs after surgery (control: 460±304 vs NTG: 762±411 ml) (p=0.03). The PaO2/FiO2 ratio was lower for patients who received NTG (control: 372±48 vs NTG: 308±106; p=0.046).

Discussion

The above-described study indicates that intravenous administration of NTG during high-risk cardiac surgery reduced or prevented hemispheric cerebral oxygen desaturation during CPB. This favorable effect could not be demonstrated using traditional measures of global perfusion, as both groups showed similar cardiac index, central jugular venous saturation, and plasma lactates. Furthermore, the results of this study showed that up to one third of patients in the placebo group suffered from significant brain oxygen desaturation. Therefore, intravenous administration of a vasodilator, such as NTG, represents an effective strategy to maintain brain oxygen regional saturation, particularly in high-risk patients undergoing cardiac surgery under CPB. More generally, these data show that intravenous administration of a vasodilator, such as NTG, represents an effective strategy to maintain brain oxygen regional saturation in any patient undergoing surgery.

In the present study, the perfusion rate of the study drug was not adjusted according to cerebral saturation values and did not have a predetermined strategy to correct cerebral oxygen desaturation if other parameters were normal. The baseline values for cerebral saturation were maintained throughout the procedure. This issue is important as the literature suggests that prolonged and/or severe desaturations during cardiac procedures, as indicated by the cerebral oximeter measures, predict a higher risk of postoperative neurological-psychological complications (20, 34). An absolute reduction of 20% under baseline value or saturation below 50% has been proposed as justification for intervention (20, 34-38), although most of the available studies suffer from methodological limitations (20).

The favorable evolution of cerebral oxygen saturation in the NTG patients was not associated with similar changes in other markers of global tissue perfusion. Several studies have shown that tissue perfusion could be impaired in the presence of “normal” hemodynamic conditions using gastric tonometry or sublingual microcirculation monitors (39). In such instance, vasodilators such as NTG, have been proposed as potential therapeutic agents (7, 40).

Higher elevation of CK-MB in the NTG group may indicate a possible side effect of the proposed treatment, but it is difficult to assess the real clinical impact of this difference. CK-MB has been shown to lack specificity for the diagnosis of perioperative myocardial infarct (41, 42). The population that was studied included many patients with valve surgery, for whom precise ischemic cut-off is even less well defined (43). As the troponins level and the ST changes were not recorded, it is hard to conclude that the patients in the NTG group really experienced more perioperative ischemic episodes. There was also a trend to worse outcome for some variables in the NTG group including the use of vasopressors for more than 24 hrs and the length of hospital stay. Baseline difference between groups such as higher right atrial and pulmonary artery pressure in the NTG group may explain this result. The bleeding complications were similar in both groups except for the blood losses during the first 24 hrs after surgery. Longest CPB may again be an explanation. Previous clinical studies have never demonstrated more clinical bleeding with NTG (44), despite its theoretical anti-platelet effect. Nitroglycerin will also dilate pulmonary vessels and this could increase intrapulmonary shunt. Accordingly, partial pressure of oxygen in the arterial blood to inspired fraction of oxygen (PaO2/FiO2 ratio) at the end of surgery was statistically lower in the NTG group. The difference was probably without any clinical consequence, as values stayed over 300 mm Hg in both groups.

In summary, NTG infusion before and during CPB appears as an effective strategy to prevent the reduction of cerebral oxygen saturation during CPB in high-risk patients undergoing complex cardiac surgery, and would also be expected to be effective in the same manner in other patients undergoing other surgeries. Surprisingly, the systematic administration of NTG to patients did not result in significant disadvantages as measured by the outcome of the surgery. Instead, administration of this vasodilator during surgery was shown to effectively maintain brain oxygen saturation levels (and thus reduce or prevent brain oxygen desaturation) in the patients, and also to reduce the total amount of NTG administered to the patients, as compared to those in the placebo group.

Although the present invention has been described hereinabove by way of preferred embodiments thereof, it can be modified without departing from the spirit, scope and nature of the subject invention, as defined in the appended claims.

TABLE 1
Baseline characteristics of the study population in a study for
assessing the effectiveness of nitroglycerin in maintaining
hemispheric cerebral oxygen saturation during surgery.

| | Control | Nitroglycerin |
|---|---|---|
| Characteristic | (n = 15) | (n = 15) |
| Age, yrs | 75 ± 9 | 71 ± 10 |
| Sex, n (%) | | |
| Male | 6 (40) | 9 (60) |
| Female | 9 (60) | 6 (40) |
| Body-mass index, kg/m² | 27 ± 4 | 26 ± 4 |
| NYHA class, n (%) | | |
| 1 | 5 (36) | 2 (15) |
| 2 | 3 (21) | 3 (23) |
| 3 | 5 (36) | 5 (39) |
| 4 | 1 (7) | 3 (23)^{a} |
| Parsonnet score | 25 ± 8 | 29 ± 9 |
| Current smoking, n (%) | 2 (13) | 1 (7) |
| Type of surgery, n (%) | | |
| One valve | 4 (27) | 4 (27) |
| Multiple valves | 1 (7) | 1 (7) |
| CABG | 3 (20) | 2 (13) |
| CABG + valve(s) | 7 (47) | 7 (47) |
| Other | 0 (0) | 1 (7) |
| Redo surgery, n (%) | 4 (27) | 6 (40) |
| Cardiac disease, n (%) | | |
| Prior myocardial infarction | 3 (20) | 2 (13) |
| Recent myocardial infarction | 3 (20) | 3 (20) |
| Unstable angina | 4 (27) | 4 (27) |
| Congestive heart failure | 9 (60) | 11 (73) |
| Acute endocarditis | 0 (0) | 2 (13) |
| Atrial fibrillation | 6 (40) | 5 (33) |
| Pacemaker | 1 (7) | 1 (7) |
| Comorbidities, n (%) | | |
| Hypertension | 11 (73) | 9 (60) |
| Diabetes mellitus | 2 (13) | 3 (20) |
| Peripheral vascular disease | 6 (40) | 5 (33) |
| Renal failure | 5 (33) | 6 (40) |
| COPD | 4 (27) | 1 (7) |
| Drug therapy at admission, n (%) | | |
| Nitrates | 4 (27) | 3 (20) |
| Calcium-channel antagonists | 6 (40) | 4 (27) |
| Beta-blockers | 7 (47) | 13 (87)^{b} |
| ACE inhibitors | 8 (53) | 8 (53) |
| Digoxin | 5 (33) | 3 (20) |
| Diuretics | 9 (60) | 12 (80) |
| Aspirin | 5 (33) | 5 (33) |
| Left ventricular ejection fraction, % | 49 ± 12 | 50 ± 12 |
| Glycemia at the beginning of surgery | 6.4 ± 1.7 | 6.9 ± 2.1 |
| Duration of surgery, mins | | |
| CPB | 97 ± 32 | 118 ± 49 |
| Aorta clamping | 72 ± 28 | 84 ± 49 |

NYHA, New York Heart Association;
CABG, coronary artery bypass graft;
COPD, chronic obstructive pulmonary disease;
ACE, angiotensin-converting enzyme;
BMI, body-mass index;
CPB, cardiopulmonary bypass.
^{a}There were no significant difference between groups, except for beta-blockers use (p = .05).
^{b}Not available in 2 patients.

TABLE 2
Mean cerebral saturation and other perfusion values during surgery for the
population of Table 1.

| Cerebral | | | | | p value | p value | p value |
|---|---|---|---|---|---|---|---|
| saturation | | T0 | T1 | T2 | (group) | (time) | (group * time) |
| Left | Control | 63 ± 8 | 61 ± 11 | 52 ± 14 | .28 | .052 | .006^{a} |
| | NTG | 54 ± 11 | 56 ± 13 | 56 ± 7 | | | |
| Right | Control | 59 ± 11 | 56 ± 14 | 46 ± 14 | .71 | .02 | .005^{a} |
| | NTG | 51 ± 8 | 53 ± 11 | 53 ± 7 | | | |
| ScVO₂ | Control | 82 ± 4 | 84 ± 5 | 78 ± 6 | .89 | .0003 | .21 |
| | NTG | 78 ± 8 | 86 ± 5 | 79 ± 7 | | | |
| ΔPCO₂ | Control | 7 ± 2 | 4 ± 3 | 4 ± 2 | .89 | <.0001 | .18 |
| | NTG | 7 ± 1 | 3 ± 2 | 5 ± 3 | | | |
| Lactates | Control | 1.4 ± 0.6 | 2.8 ± 1.0 | 3.2 ± 1.3 | .16 | <.0001 | .41 |
| | NTG | 1.5 ± 0.5 | 3.2 ± 0.8 | 4.0 ± 1.9 | | | |

NTG, nitroglycerin;
T0, baseline value before nitroglycerin infusion;
T1, beginning of cardiopulmonary bypass;
T2, end of cardiopulmonary bypass times;
ScVO₂, central venous blood saturation provided by the distal port of the Swan-ganz catheter;
ΔPCO₂, difference between partial pressure of carbon dioxyde of arterial and venous blood.
^{a}T0 and T1 are statistically different from T2, but only in control group.

TABLE 3
Main hemodynamic values during surgery for the population of
Table 1

| | | | | | | | p |
|---|---|---|---|---|---|---|---|
| | | | | | | | value |
| Hemodynamic | | | | | p value | p value | (group |
| variables | | T0 | T1 | T2 | (group) | (time) | *time) |
| Systolic BP | Control | 109 ± 16 | 101 ± 15 | 109 ± 20 | .41 | .053 | .95 |
| | NTG | 105 ± 21 | 95 ± 17 | 106 ± 15 | | | |
| Heart rate | Control | 53 ± 11 | 59 ± 11 | 70 ± 11 | .08 | <.0001^{a} | .83 |
| | NTG | 55 ± 9 | 61 ± 15 | 78 ± 15 | | | |
| RAP | Control | 10 ± 3 | 10 ± 5 | 12 ± 5 | .03 | .01^{a} | .42 |
| | NTG | 13 ± 5 | 12 ± 6 | 17 ± 3 | | | |
| Systolic PAP | Control | 32 ± 6 | 32 ± 7 | 37 ± 8 | .004 | .0006^{a} | .16 |
| | NTG | 44 ± 18 | 37 ± 10 | 48 ± 10 | | | |
| PAWP | Control | 15 ± 4 | 15 ± 4 | 20 ± 4 | .35 | .06 | .77 |
| | NTG | 18 ± 7 | 15 ± 9 | 22 ± 3 | | | |
| Indexed | Control | 2.0 ± 0.3 | 1.9 ± 0.4 | 2.2 ± 0.4 | .70 | .0003^{a} | .43 |
| cardiac output | NTG | 1.9 ± 0.4 | 1.9 ± 0.4 | 2.4 ± 0.8 | | | |

BP, blood pressure;
NTG, nitroglycerin;
RAP, right atrial pressure;
PAP, pulmonary artery pressure;
PAWP, pulmonary artery wedge pressure;
T0, baseline value before nitroglycerin infusion;
T1, beginning of cardiopulmonary bypass;
T2, end of cardiopulmonary bypass times.
^{a}T0 and T1 are statistically different from T2.

TABLE 4
Vasopressors and fluids needs for the population of Table 1

| | Control | NTG | p value |
|---|---|---|---|
| Vasopressors^{a} | | | |
| Norepinephrine, μg | 546 ± 563 | 1209 ± 1037 | .04 |
| Norepinephrine, μg/min^{b} | 2.2 ± 2.4 | 4.2 ± 3.7 | .096 |
| Neosinephrine, μg | 6330 ± 3931 | 11,303 ± 8910 | .06 |
| Neosinephrine, μg/min^{b} | 31 ± 20 | 36 ± 27 | .55 |
| Milrinone, μg | 0 (0; 5300) | 0 (0; 6800) | .64 |
| Milrinone (ug/min | 0 (0; 17.21) | 0 (0; 23.53) | 0.5415 |
| Vasopressin, U | 2 + 4 | 3 + 4 | .36 |
| Vaso (ug/min) | 0.0074 + 0.0122 | 0.0116 + 0.0137 | 0.3837 |
| Ephedrine, mg | 3.5 + 7.9 | 4.3 ± 8.8 | .78 |
| Ephedrine (ug/min) | 18.85 + 42.93 | 14.73 + 29.59 | 0.7618 |
| IV fluids during surgery, | 4972 ± 1175 | 5582 ± 1322 | .19 |
| mL | | | |

NTG, nitroglycerin.
^{a}Three patients also received epinephrine as “salvage therapy”, one in the nitroglycerin group and 2 in the placebo group.
^{b}Mean dose per minute for total surgery duration.

TABLE 5
Other clinical and security outcomes for the population of Table 1

| | Control | NTG | p value |
|---|---|---|---|
| CK-MB^{a} | 19 ± 12 | 58 ± 67 | .006 |
| Lactates, mEq/L | 1.7 ± 0.8 | 2.6 ± 2.8 | .27 |
| Post-CPB hemodynamic | 10 (67) | 11 (73) | .69 |
| instability, n (%) | | | |
| IABP, n (%) | 0 (0) | 1 (7) | N/A |
| Vasopressors use >24 hrs, n (%) | 4 (27) | 8 (53) | .14 |
| ICU stay, days | 3 ± 2 | 5 ± 4 | .18 |
| Hospital stay, days | 9 ± 3 | 14 ± 7 | .06 |
| Death, n (%) | 0 (0) | 2 (13) | N/A |
| Blood loss, mL | | | |
| During surgery^{b} | 429 ± 261 | 547 ± 251 | .23 |
| First 24 hrs | 460 ± 304 | 762 ± 411 | .03 |
| Heparin, mg | 306 ± 118 | 393 ± 111 | .047 |
| Heparin, mg/duration of CPB | 3.48 ± 1.73 | 3.86 ± 1.92 | .569 |
| Blood units transfused, mL | 332 ± 408 | 380 ± 400 | .75 |
| PaO₂/FiO₂ ratio | 372 ± 48 | 308 ± 106 | .046 |

NTG, nitroglycerin;
CPB, cardiopulmonary bypass;
IABP, intra-aortic balloon pump;
CK, creatine kinase;
ICU, intensive care unit;
N/A, not available because of small number of events;
U, units of vasopressin;
P/F ratio, ratio of the partial pressure of oxygen in the arterial blood to inspired fraction of oxygen at the end of surgery.
^{a}The CK-MB log value was analyzed because the value did not have a normal distribution.
^{b}There was no statistically significant difference between the change in hemoglobin before and after surgery (p = 0.17).

### LIST OF REFERENCES

1. Wan S, LeClerc J L, Vincent J L: Inflammatory response to cardiopulmonary bypass: mechanisms involved and possible therapeutic strategies. Chest 1997; 112:676-692
2. Asimakopoulos G, Smith P L, Ratnatunga C P, et al: Lung injury and acute respiratory distress syndrome after cardiopulmonary bypass. Ann Thorac Surg 1999; 68:1107-1115
3. Frass O M, Buhling F, Tager M, et al: Antioxidant and antiprotease status in peripheral blood and BAL fluid after cardiopulmonary bypass. Chest 2001; 120:1599-1608
4. Arrowsmith J E, Grocott H P, Reves J G, et al: Central nervous system complications of cardiac surgery. Br J Anaesth 2000; 84:378-393
5. Roach G W, Kanchuger M, Mangano C M, et al: Adverse cerebral outcomes after coronary bypass surgery. Multicenter Study of Perioperative Ischemia Research Group and the Ischemia Research and Education Foundation Investigators. N Engl J Med 1996; 335:1857-1863
6. Newman M F, Kirchner J L, Phillips-Bute B, et al: Longitudinal assessment of neurocognitive function after coronary-artery bypass surgery. N Engl J Med 2001; 344:395-402
7. Goldman S, Sutter F, Ferdinand F, et al: Optimizing intraoperative cerebral oxygen delivery using noninvasive cerebral oximetry decreases the incidence of stroke for cardiac surgical patients. Heart Surg Forum 2004; 7:E376-E381
8. Lefer D J, Nakanishi K, Johnston W E, et al: Antineutrophil and myocardial protecting actions of a novel nitric oxide donor after acute myocardial ischemia and reperfusion of dogs. Circulation 1993; 88:2337-2350
9. Bilinska M, Maczewski M, Beresewicz A: Donors of nitric oxide mimic effects of ischaemic preconditioning on reperfusion induced arrhythmias in isolated rat heart. Mol Cell Biochem 1996;160-161: 265-271
10. Banerjee S, Tang X L, Qiu Y, et al: Nitroglycerin induces late preconditioning against myocardial stunning via a PKC-dependent pathway. Am J Physiol 1999; 277:H2488-H2494
11. Kawashima M, Bando T, Nakamura T, et al: Cytoprotective effects of nitroglycerin in ischemia-reperfusion-induced lung injury. Am J Respir Crit Care Med 2000; 161:935-943
12. Dun Y, Hao Y B, Wu Y X, et al: Protective effects of nitroglycerin-induced preconditioning mediated by calcitonin gene-related peptide in rat small intestine. Eur J Pharmacol 2001; 430:317-324
13. Thangathurai D, Charbonnet C, Wo C C, et al: Intraoperative maintenance of tissue perfusion prevents ARDS. Adult Respiratory Distress Syndrome. New Horiz 1996; 4:466-474
14. Kim M B, Ward D S, Cartwright C R, et al: Estimation of jugular venous O2 saturation from cerebral oximetry or arterial O2 saturation during isocapnic hypoxia. J Clin Monit Comput 2000; 16:191-199
15. Madsen P L, Secher N H: Near-infrared oximetry of the brain. Prog Neurobiol 1999; 58:541-560
16. Edmonds H L Jr, Rodriguez R A, Audenaert S M, et al: The role of neuromonitoring in cardiovascular surgery. J Cardiothorac Vasc Anesth 1996; 10:15-23
17. Dujovny M, Misra M, Widman R: Cerebral oximetry—techniques. Neurol Res 1998; 20 (Suppl 1):S5-12
18. Edmonds H L Jr: Multi-modality neurophysiologic monitoring for cardiac surgery. Heart Surg Forum 2002; 5:225-228
19. Bernstein A D, Parsonnet V: Bedside estimation of risk as an aid for decision-making in cardiac surgery. Ann Thorac Surg 2000; 69:823-828
20. Taillefer M C, Denault A Y: Cerebral near-infrared spectroscopy in adult heart surgery: systematic review of its clinical efficacy. Can J Anaesth 2005; 52:79-87
21. Coriat P, Daloz M, Bousseau D, et al: Prevention of intraoperative myocardial ischemia during noncardiac surgery with intravenous nitroglycerin. Anesthesiology 1984; 61:193-196
22. Curry S C, Arnold-Capell P: Toxic effects of drugs used in the ICU. Nitroprusside, nitroglycerin, and angiotensin-converting enzyme inhibitors. Crit Care Clin 1991; 7:555-581
23. Bojar R M, Rastegar H, Payne D D, et al: Methemoglobinemia from intravenous nitroglycerin: a word of caution. Ann Thorac Surg 1987; 43:332-334
24. Flaherty J T, Magee P A, Gardner T L, et al: Comparison of intravenous nitroglycerin and sodium nitroprusside for treatment of acute hypertension developing after coronary artery bypass surgery. Circulation 1982; 65:1072-1077
25. Kaplan J A, Dunbar R W, Jones E L: Nitroglycerin infusion during coronary-artery surgery. Anesthesiology 1976; 45:14-21
26. Kaplan J A, Jones E L: Vasodilator therapy during coronary artery surgery. Comparison of nitroglycerin and nitroprusside. J Thorac Cardiovasc Surg 1979; 77:301-309
27. Fung H L, Chung S J, Bauer J A, et al: Biochemical mechanism of organic nitrate action. Am J Cardiol 1992; 70:4B-10B
28. Schafer A I, Alexander R W, Handin R I: Inhibition of platelet function by organic nitrate vasodilators. Blood 1980; 55:649-654
29. Thomson I R, Mutch W A, Culligan J D: Failure of intravenous nitroglycerin to prevent intraoperative myocardial ischemia during fentanyl-pancuronium anesthesia. Anesthesiology 1984; 61:385-393
30. Gallagher J D, Moore R A, Jose A B, et al: Prophylactic nitroglycerin infusions during coronary artery bypass surgery. Anesthesiology 1986; 64:785-789
31. Lell W, Johnson P, Plagenhoef J, et al: The effect of prophylactic nitroglycerin infusion on the incidence of regional wall-motion abnormalities and ST segment changes in patients undergoing coronary artery bypass surgery. J Card Surg 1993; 8:228-231
32. Apostolidou I A, Despotis G J, Hogue C W Jr, et al: Antiischemic effects of nicardipine and nitroglycerin after coronary artery bypass grafting. Ann Thorac Surg 1999; 67:417-422
33. Zvara D A, Groban L, Rogers A T, et al: Prophylactic nitroglycerin did not reduce myocardial ischemia during accelerated recovery management of coronary artery bypass graft surgery patients. J Cardiothorac Vasc Anesth 2000; 14:571-575
34. Yao F S, Tseng C C, Ho C Y, et al: Cerebral oxygen desaturation is associated with early postoperative neuropsychological dysfunction in patients undergoing cardiac surgery. J Cardiothorac Vasc Anesth 2004; 18:552-558
35. Edmonds H L Jr: Advances in neuromonitoring for cardiothoracic and vascular surgery. J Cardiothorac Vasc Anesth 2001; 15:241-250
36. Edmonds H L Jr, Cao L, Yu Q J: In the elderly, coronary artery bypass grafting associated with more brain O2 desaturation and cognitive dysfunction. Ann Thorac Surg 2002; 73:S375
37. Cho H, Nemoto E M, Yonas H, et al: Cerebral monitoring by means of oximetry and somatosensory evoked potentials during carotid endarterectomy. J Neurosurg 1998; 89:533-538
38. Samra S K, Dy E A, Welch K, et al: Evaluation of a cerebral oximeter as a monitor of cerebral ischemia during carotid endarterectomy. Anesthesiology 2000; 93:964-970
39. Buwalda M, Ince C: Opening the microcirculation: can vasodilators be useful in sepsis? Intensive Care Med 2002; 28:1208-1217
40. Harvey L, Edmonds H L Jr, Ganzel B L, et al: Cerebral oximetry for cardiac and vascular surgery. Semin Cardiothorac Vasc Anesth 2004; 8:147-166
41. Benoit M O, Paris M, Silleran J, et al: Cardiac troponin I: its contribution to the diagnosis of perioperative myocardial infarction and various complications of cardiac surgery. Crit Care Med 2001; 29:1880-1886
42. Bimmel D, Patermann B, Schlosser T, et al: Do we still need CK-MB in coronary artery bypass grafting surgery? J Cardiovasc Surg (Torino) 2003; 44:191-196
43. Jarvinen A, Mattila T, Kyosola K: Serum CK-MB isoenzyme after aortic and mitral valve replacements. Ann Clin Res 1983; 15:189-193
44. Williams H, Langlois P F, Kelly J L: The effect of simultaneous intravenous administration of nitroglycerin and heparin on partial thromboplastin time. Mil Med 1995; 160:449-452.

All references cited and/or discussed in this specification are incorporated herein by reference in their entirety and to the same extent as if each reference was individually incorporated by reference.

What is claimed is:

1. A method for reducing or preventing hemispheric cerebral oxygen desaturation in a subject undergoing surgery, said method comprising the prophylactic administration of a vasodilator to the subject.

2. The method as defined in claim 1, wherein said vasodilator is administered in an amount sufficient for maintaining hemispheric cerebral oxygenation above a predetermined oxygen saturation level.

3. The method as defined in claim 2, wherein said predetermined oxygen saturation level is equal to about 75 percent of an hemispheric cerebral oxygenation in said subject prior to the administration of said vasodilator and prior to the beginning of said surgery.

4. The method as defined in claim 1, wherein said vasodilator is nitroprusside.

5. The method as defined in claim 1, wherein said vasodilator is nitroglycerine.

6. The method as defined in claim 5, wherein said nitroglycerine is administered intravenously into the subject.

7. The method as defined in claim 6, wherein said nitroglycerine is administered through one of intravenous injection and infusion.

8. A method as defined in claim 6, wherein said nitroglycerine is administered prior to performing the surgery.

9. A method as defined in claim 6, wherein said nitroglycerine is injected both prior to performing said surgery and during at least a portion of the duration of said surgery.

10. A method as defined in claim 9, wherein said nitroglycerine is injected both prior to performing said surgery and during the entire duration of said surgery.

11. The method as defined in claim 6, wherein said nitroglycerine is injected at a rate of about 0.001 μg/kg of subject weight/min to about 100 μg/kg of subject weight/min.

12. The method as defined in claim 6, wherein said nitroglycerine is injected at a rate of about 0.1 μg/kg of subject weight/min to about 5 μg/kg of subject weight/min.

13. The method as defined in claim 6, wherein said nitroglycerine is injected at a rate of about 0.5 μg/kg of subject weight/min to about 1 μg/kg of subject weight/min.

14. The method as defined in claim 1, wherein said surgery involves extra-corporal circulation.

15. The method as defined in claim 14, comprising:
administering said vasodilator in the bloodstream of said subject at a first rate before the beginning of said extra-corporal circulation; and
administering said vasodilator in the bloodstream of said subject at a second rate after the beginning of said extra-corporal circulation.

16. The method as defined in claim 13, wherein said second rate is substantially larger than said first rate.

17. The method as defined in claim 1, wherein said subject is a mammal.

18. The method as defined in claim 15, wherein said mammal is a human.

19. The method as defined in claim 1, wherein said vasodilator is a nitric oxide donor.

20. The method as defined in claim 1, comprising said prophylactic administration of said vasodilator to said subject in an amount sufficient for preventing hemispheric cerebral oxygen desaturation.
